(19) United States
(12) Patent Application Publication
(10) Pub. No.: US 2005/0214737 A1
(43) Pub. Date: Sep. 29, 2005

(54) Transparent filtered capillaries

(76) Inventors: Matthew J. Dejneka, Corning, NY (US); Michael H. Rasmussen, Millport, NY (US); Po Ki Yuen, Corning, NY (US)

Correspondence Address: CORNING INCORPORATED, SP-TI-3-1, CORNING, NY, 14831, US

(21) Appl. No.: 10/809,956

(22) Filed: Mar. 26, 2004

(51) Int. Cl. C12Q 1/00; C12M 1/34

(52) U.S. Cl. 435/4; 435/287.1

(57) ABSTRACT

A microfluidic reactor (10) for trapping one or more particles of predetermined nominal size or range of sizes that have entered a flow inlet (12) includes a transparent reaction zone (14) which also serves as an in-situ detection zone wherein the detection zone is arranged so as substantially to correspond in shape to an optical detector (456). A porous filter (16) having a plurality of holes (160) being smaller than the nominal size or range of sizes of the particles (200) are arranged so as to trap the particles in the reaction zone (14) while a fluid (18) flows from the flow inlet (12) through the reaction zone (14) and the filter (16).

### BACKGROUND OF THE INVENTION

1. Field of the Invention

The present invention relates generally to high throughput biological assay devices, and particularly to capillary tubes in high throughput biological particle-based assay devices.

2. Technical Background

Particle-based assays are known. With particle-based assays, biomolecular reactions take place either on the surface of microscopic beads called microspheres or microscopic bars called microrods (typical sizes are in the submicron and micron ranges). In order to use the particle-based assays to study the biomolecular reactions, for each reaction, a number of molecules are first immobilized or attached to the surface of the particles. These attached molecules are typically called probes. A sample solution containing target molecule(s) is applied to each well or tube and mixed with the “treated” particles. A target or analyte presented in the sample reacts with the probe molecules. In general, the target molecules or a reference molecule co-exited with the targets in the sample are tagged with an optically active compound, of which the fluorescence or luminescence is increased during the reaction between the target molecules (or the reference molecules) and the probe molecules, for example. A qualitative and/or quantitative analysis of the composition of the sample fluid can thus be carried out by illuminating and optically scanning the contents of the wells. In a multiplex assay format, the particles are either internally color-coded or are coded with different predefined color or reflective pattern, as in a color stripe of a bar-coded rod or are synthesized with built-in infrared (IR) and Raman spectroscopic barcodes such that multiple reactions can be performed in a single tube or well. In this case, there are two sources of information, the predefined pattern inside the particles to identify the type of reaction and the reporter color on the surface of the particles to signal the magnitude of the biomolecular reaction.

Particle-based assays typically consist of multiple wash cycles, different phases of incubations, and data analysis. Among other complex methods, the assays are typically performed either in filter-bottomed microplates, such as available from Millipore Corporation, Bedford, Mass., as part Cat. #MABVN-1210 or in centrifuge tubes. When using the filter-bottomed microplate, wash cycles and incubations are carried out in each well of the microplate. Reagent or sample (target molecules) is added to each well containing the particles and after each step, the solution is removed from wells via vacuum using a filtration manifold (e.g., Millipore Corporation, Bedford, Mass., Cat. # MAVM09601). The wash and incubation are repeated until the assay is completed. If centrifuge tubes are used, washing is performed manually by first centrifuging the particles in tubes followed by completely aspirating the solution from the tubes which is done by gently lowering an aspiration tip (aspiration device) into the bottom of each tube. Care has to be taken not to aspirate the particle pellet. After aspiration, the wash and incubation are repeated until the assay is completed. In a multiplex assay format, different particles from different wells or tubes are removed and mixed into a single tube or well after the first phase of washing and incubation. Then, additional wash cycles and incubations are performed to complete the assay. In both approaches, the multiple wash cycles and incubations are labor intensive and particle loss is a concerned when performing particle-based assays.

At the completion of the assay, to analyze the test result, the particle mixture is removed from the wells or tubes and is injected into a flow cytometer that aligns the particles in a single file where lasers illuminate the colors on the surface of each particle. In the case where there are predefined pattern inside the particles, a custom-made instrument (such as a modified flow cytometer) is required with an extra laser illuminates the pattern inside the particles to identify the type of reaction. Next, advanced optics capture the color signals. Finally, digital signal processing translated the signals into real-time, quantitative data for each reaction. Alternatively, particles are isolated and dried from the particle mixture. They are then scanned with a scanner and/or imaged with an optical microscope for data analysis. Clearly, both techniques require additional handling and transferring of the particle mixture. It may lead to particle loss and hence a large number of particles may be required. In the drying technique, it is very difficult to prevent particles from stacking to form a monolayer which will affect data analysis. Also, complicated fluid handling is required in such conventional techniques which do not provide an efficient method of preparing the particles for imaging or scanning for data analysis.

Low-pressure filter assemblies are available in the market. However, the main disadvantage of the existing products is that they are not transparent which will prevent the data analysis of the particles through scanning and imaging. Also, low-pressure filter assemblies are made from polymer which may not be biocompatible and may not be able to resist heat or solvents. Therefore, a tool for conveniently performing particle-based assays with minimal sample, minimal particle loss or human handling is desired.

Furthermore, genetic testing is another area that the present invention is intended for. Two key steps in genetic testing procedures, cell isolation and nucleic acid amplification reactions, have been demonstrated in a computer numerical control-machined Plexiglas-based microchip module comprising of a custom-made heater-cooler for thermal cycling, a series of microchannels for transporting human whole blood and reagents in and out of a dual-purpose glass-silicon microchip (Yuen et al., Genomic Research, 2001, 11, 405-412). The cell isolation and polymerase chain reaction (PCR) were preformed inside the dual-purpose glass-silicon microchip containing a series of 3.5 m feature-sized weir-type filters, formed by an etched silicon dam spanning the flow chamber. Although the microchip module was demonstrated to be an effective tool for integrating the cell isolation and PCR, it requires laborious steps of fabricating the glass-silicon microchips which have to be fabricated in a clean room environment. Also, each component of the microchip module has to be fabricated separately and then assembled together before use. Thus, an alternative method to the microchip module that can overcome the shortcomings of the microchip module would be valuable and attractive.

### SUMMARY OF THE INVENTION

One aspect of the invention is a microfluidic reactor for trapping one or more particles of predetermined nominal size or range of sizes that have entered a flow inlet which includes a transparent reaction zone for also serves as an in-situ detection zone wherein the detection zone is arranged so as substantially to correspond in shape to an optical detector. A porous filter having a plurality of holes being smaller than the nominal size or range of sizes of the particles are arranged so as to trap the particles in the reaction zone while a fluid flows from the flow inlet through the reaction zone and the filter.

In another aspect, the present invention includes the integration of a plurality of smaller capillaries to a larger capillary to form a filtered capillary tube as the microfluidic reactor.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention, and together with the description serve to explain the principles and operations of the invention.

### BRIEF DESCRIPTION OF THE DRAWINGS

FIG. 1 is a perspective view of one embodiment of the present invention;

FIG. 2 is a cross-sectional view of another embodiment of the filter 16 of FIG. 1, according to the invention;

FIG. 3 is a side-view of a process for integrating the filter 16 of FIG. 2 inside a capillary tube to form another embodiment of the microfluidic reactor 10 of FIG. 1, according to the invention;

FIG. 4 is a parallel array of the bundled arrangement of the microfluidic reactors 10 of FIG. 1, according to the invention;

FIG. 5 is a vertical automated system using the bundled arrangement of the microfluidic reactors 10 of FIG. 4, according to the invention;

FIG. 6 is a horizontal automated system using the bundled arrangement of the microfluidic reactors 10 of FIG. 4, according to the invention;

FIGS. 7-9 are representations of the steps involved when performing a multiplex assay with the filtered tube 10 of FIG. 1, according to the invention;

FIGS. 10-12 are representations of the steps involved when performing white blood cells isolation with the filtered tube 10 of FIG. 1, according to the invention; and

FIG. 13 is a schematic of a temperature control module for the filtered tube 10 of FIGS. 10-12 in a side view, with the filtered tube 10, and a top view without the filtered tube 10, according to the invention.

### DETAILED DESCRIPTION OF THE PREFERRED EMBODIMENTS

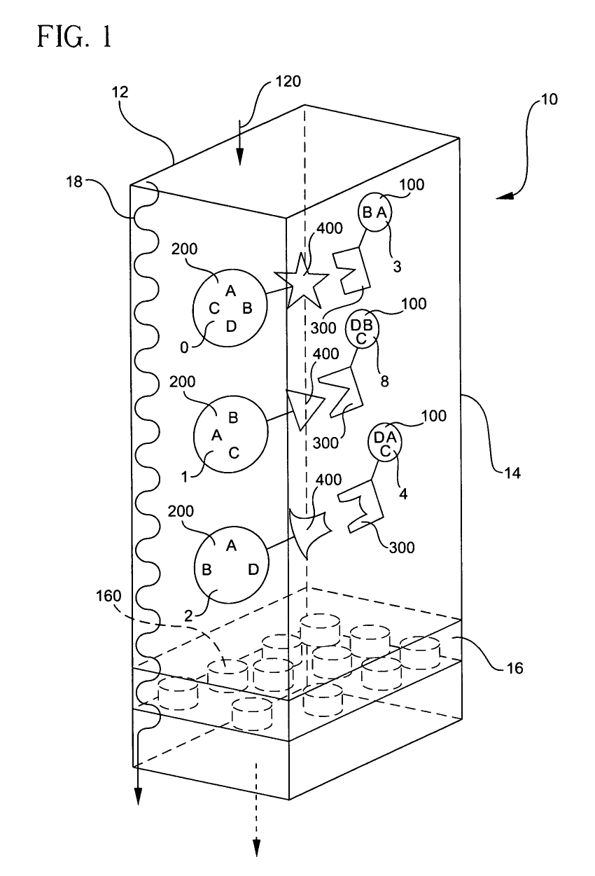

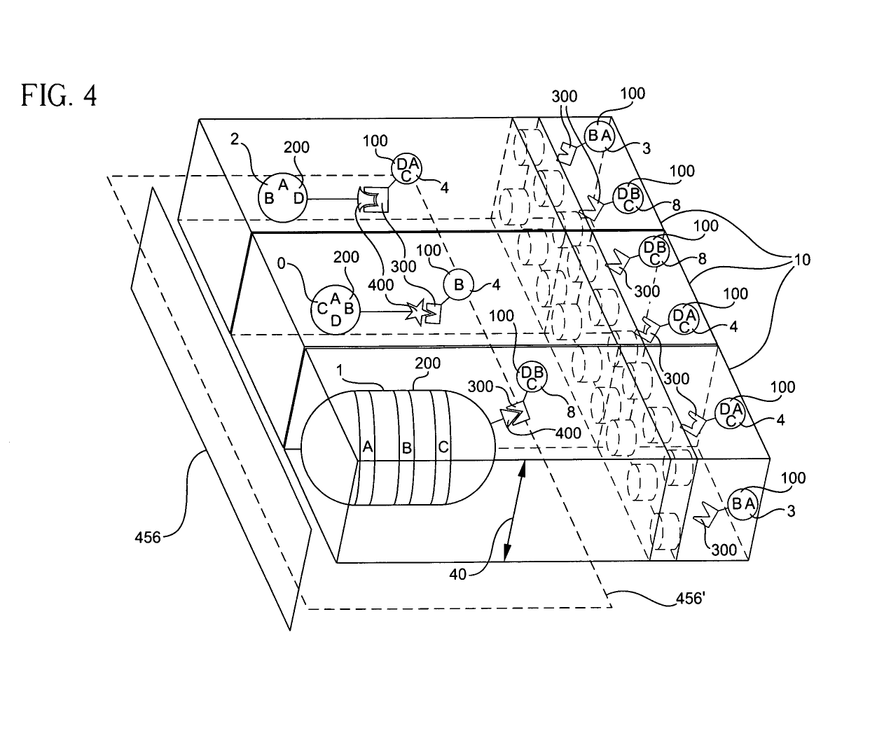

Reference will now be made in detail to the present preferred embodiments of the invention, examples of which are illustrated in the accompanying drawings. Whenever possible, the same reference numerals will be used throughout the drawings to refer to the same or like parts. One embodiment of the microfluidic reactor of the present invention is shown in FIG. 1, and is designated generally throughout by the reference numeral 10. In accordance with the invention, the present invention for a method and apparatus of microfluidic reaction includes a first element or step of trapping one or more particles of predetermined nominal size or range of sizes that have entered a flow inlet 12. A transparent reaction zone 14 serves as an in-situ detection zone wherein the detection zone is arranged so as substantially to correspond in shape to an optical detector 456, as represented in FIGS. 4, 5, and 6. A porous filter 16 having a plurality of holes 160 being smaller than the nominal size or range of sizes of the particles 200 are arranged so as to trap the particles in the reaction zone 14 while a fluid 18 flows from the flow inlet 12 through the reaction zone 14 and the filter 16. As embodied herein and depicted in FIG. 1, the reaction zone 14 can be formed of a transparent capillary, made from glass, polymers, or another suitable material that can be coated with a suitable solvent resistance.

Both the inside and outside surfaces of the capillary tube can be of any suitable shapes to provide an imageable surface for a detector. For example, a circular, square or rectangular inner channel can be provided by a capillary tube to form the reaction zone 14 that is integrated with the porous filter 16. Preferably, the cross-sectional shape of the capillary tube is either square or rectangular, both inside and outside to correspond with the shape of the detector 456 used in FIGS. 4, 5, and 6. The advantage of using a square or rectangular capillary tube rather than a circular capillary tube is that particles can be easily scanned and imaged for data analysis. A rectangular capillary filter tube, as seen in FIG. 1, also increases the surface area for imaging the particles. The porous filter 16 extends laterally across the reaction zone 14. The flow inlet 12 defines a flow axis 120 and the filter 16 intersects the flow axis 120 so as to form a porous reaction chamber by integrating the filter with the capillary to provide a filtered capillary tube or microfluidic reactor 10.

Within the filtered capillary tube for providing the microfluidic reactor 10, particle-based assays involving multiple wash cycles, different phases of incubations, and data analysis can be conveniently performed with minimal particle loss and human handling. Examples of possible particle-based assays include DNA hybridization, immunoassay, enzyme\substrate activity, etc.

A single rectangular capillary filtered tube is shown for simplicity in FIG. 1. Miniature micro to nano-sized particles 100 or 200 are disposed inside the filtered capillary tube as detectable nanosized labels 100 for use as an identifier for an analyte, sample, or target 300 or as carriers, substrates, or microsized support beads 200 for attaching a unique functional group or probe 400. Even though a line is shown representing the strong binding of either the support bead 200 to the probe 400 or the nanosized label 100 to the target 300, it is to be appreciated that no actual chemical linkage needs to be formed, but rather a physical adsorption can be present to form the binding, in the form of a functional group, for example. However, the functional group can be part of the probe 400, the target 300, the support bead 200, the nanosized label 100, or the line representing their binding.

Instead of microbeads, the particles 200 could also be cells, such as blood cells or other biological molecules to be analyzed. In this case, the capillary filtered tube can be used to isolate white blood cells from the human whole blood where the red blood cells will pass through the filter during the isolation (Yuen et al., Genomic Research, 2001, 11, 405-412). Then, polymerase chain reaction (PCR) can be performed with the white blood cells inside the capillary filtered tube. Thus, another embodiment of the present invention is in the field of genetic testing for performing cell isolation and nucleic acid amplification reactions, as one type of possible analysis.

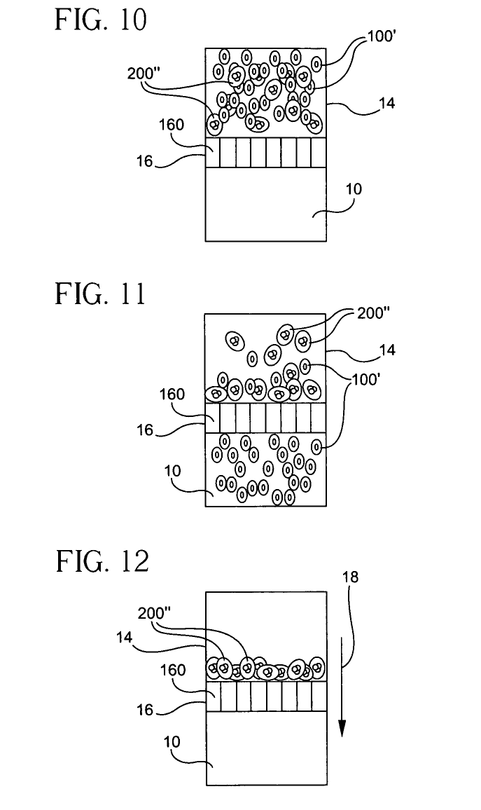

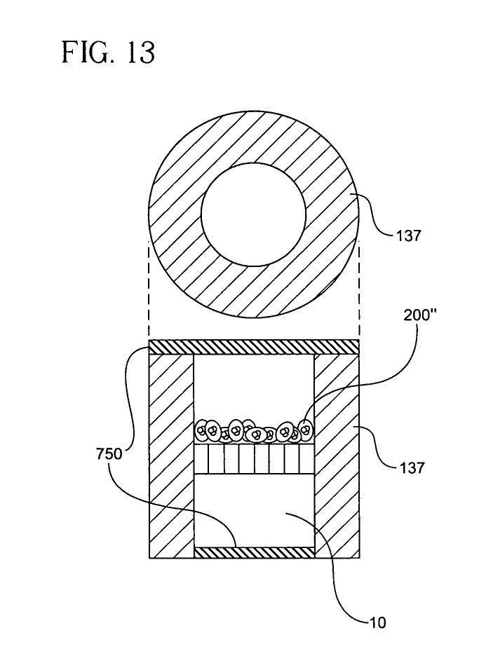

Referring to FIGS. 10-13, particles, in the form of white blood cells 200″, are first isolated from a small volume of human whole blood (e.g., less than few microliter) in the filtered capillary 10 containing a series of micron feature-sized filters (e.g., 3.5 μm) or apertures 160 to filter out the smaller red blood cells 100′. A genomic target is subsequently directly amplified by PCR on DNA released from white blood cells isolated on the filter section or the reaction zone 14 of the capillary 10 for performing biological analysis. This can be achieved by first priming the filtered capillary with phosphate buffered saline to ensure that there is no air remaining inside the filtered capillary. Then a small volume of human whole blood is injected into the filtered capillary 10. Directly following the injection of the whole blood, PCR assay mixture containing the genetic target is injected through the filtered capillary to complete the cell isolation process. Finally, the filtered capillary undergoes thermal cycling inside a temperature control module, as with a heating element 137 applied to the reaction zone 14, with each end of the filtered capillary 10 sealed with a pair of rubber gaskets 750 to prevent evaporation of the PCR assay mixture during thermal cycling. Finally, the PCR product can be recovered for detection.

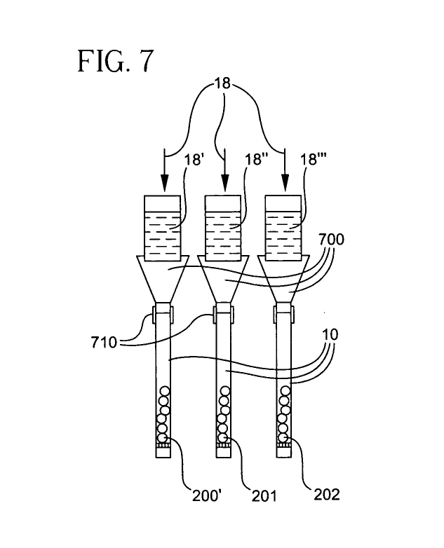

The particles 100 or 200 are homogeneously doped or spatially patterned with various combinations of rare earth (RE) elements A, B, C, or D, for example, in a glass or ceramic host to provide the codes 0, 1, 2, 4, or 8 for example to form the final particles 200′, 201, and 202 in FIG. 7 for the codes 0, 1, and 2, respectively. A spatially striped or otherwise pattern rod is seen as the particle 200 marked 1 in FIG. 4. Rare earth doped glasses are preferred for the carrier beads because of their narrow emission bands, high quantum efficiencies, noninterference with common fluorescent labels, and inertness to most organic and aqueous solvents. The particles 100 or 200 can be used as carrier beads to bind with the probe 400 or target 300, respectively for later fluorescence optical detection. Thus, the particle 100 can be used as a RE target label and the particle 200 can be used as a RE probe carrier. Also, the particle 100 can be used as a RE target label with a conventional probe. Moreover, a conventional target label, such as a common fluorescent molecular dye label, can be used with the particle 200 as a RE probe carrier, such as the final particles 200′, 201, and 202 in FIG. 7.

The porous filter can be made of glass, polymers, metal or any other material as long as the material is porous to allow fluids to flow through but captures particles. The plurality of holes can be of any shape, such as rectangular, hexagonal, circular, square, etc, patterned or randomized. Normally, the size of the filter is designed small enough to block the flow of the larger microsized particle 200. But in other applications, the filter can be made even smaller to capture the smaller nanosized particle 100.

Instead of being rectangular as shown in FIG. 1, the capillary tube forming the reaction zone 14 can be circular to accommodate a circular porous filter.

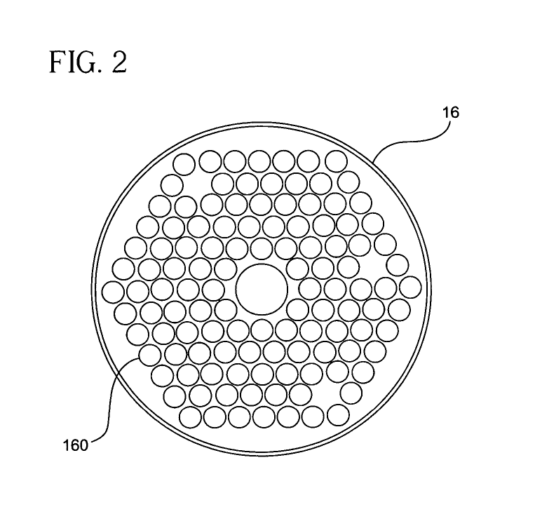

Referring to FIG. 2, a cross-section of a possible porous filter 16 is shown. As embodied herein and depicted in FIG. 2, the porous filter 16 includes a part of a microstructured fiber. The microstructured fiber (e.g., Corning Photonic Crystal Fiber with 20 μm holes) can be a drain or filtration system using any other types of microstructures with a hole size smaller than the size of the particles 200 of FIG. 1. An outer diameter of 525 μm was used as the diameter of the photonic crystal fiber having holes of a maximum diameter of 20 μm holes, separated by a pitch or spacing of 21.4 μm to provide a void filling fraction or volume ratio diameter over pitch D/P of about 0.94. The process for making such photonic crystal fibers is known by inserting and stacking smaller capillaries into a tubing or sleeve. The pitch need not be regular as long as each of the holes is smaller than the smallest dimension of the particle.

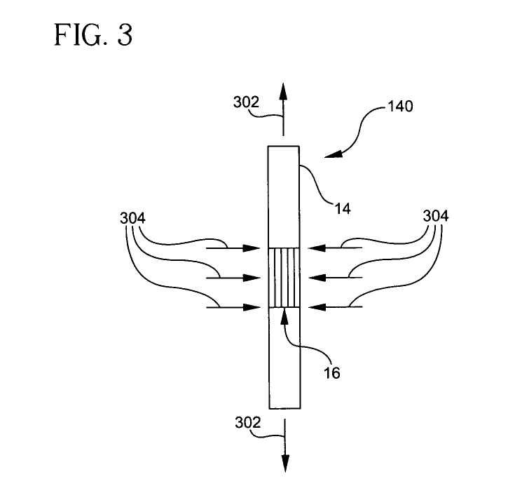

Referring to FIG. 3, a fused and collapsed process to fabricate a single filtered capillary tube for the microfluidic reactor 10 is shown, as an exemplary process. The filter 16 having the cross section as shown in FIG. 2 is fused inside a capillary tube 140 through heating for providing a section of the capillary tube as the reaction zone 14. A short piece of microstructured fiber, such as the one in FIG. 2, which is preferably as short as possible (e.g., <3 mm) to reduce the pressure drop during filtration, is first placed inside a single capillary tube. The outer diameter of the circular capillary tube is about 2.65 mm with an inside diameter of about 0.54 mm. However, the dimensions of the capillary tube and the microstructed fiber can be varied depending on the application. Then, vacuum 302 is applied at both ends of the capillary tube. Next, heat 304 is applied to the outer surface of the capillary tube at the location of the microstructured fiber. The localized heating can be performed using a ring or similarly shaped burner in order to heat the capillary evenly from all directions where the microstructured fiber is located. Due to the presence of the vacuum, the capillary tube where it is heated will collapse until it touches and fuses to the outer surface of the microstructured fiber. The piece of microstructured fiber may be longer or shorter than the collapse region.

Another method of production is to insert a longer piece of microstructured fiber that would extend from one or both ends of the capillary tube. The fiber could be scribed at one or more locations, allowing it to be easily and cleanly broken after the collapse process, leaving only a short piece fused inside the tube. The pressure inside the microstructured fiber could be placed under the same vacuum as that inside of the tube, or its pressure could be varied.

Prototypes were fabricated using Corning's Photonic Crystal Fiber with an outer diameter of 525 μm, 20 μm holes and 21.4 μm pitch as the filter 16 fused inside a glass capillary tube with an outer diameter of 2.65 mm and an inner diameter of 540 μm. The capillary tube was collapsed using a Methane/Oxygen flame from a 12-port, 5/16″ ring burner and a 15″ Hg vacuum, producing a 3-4 mm collapse region where the tube is fused to the fiber. The periphery of the integrated filtered capillary tube can be left circular or sliced or otherwise shaped to form a rectangle or square for easier stacking and/or easier imaging.

The prototype capillary integrated filter was tested with 10-30 μm glass beads available from Polysciences, Inc., Warrington, Pa., as part Catalogue #07668 as the particles. The result indicated that it is feasible to perform numerous wash cycles and incubations with minimal loss of the glass beads. Also, by injecting a solution on the opposite end, the filtered end, of the capillary filter tube, it is possible to recover the glass beads with minimal loss. This is important when one performs a multiplex assay where different beads have to be treated separately first and then they are recovered and combined into a single tube for further incubations and washes, as seen in FIGS. 7-9. Advantageously, the “photonic crystal fiber” filter provides high efficiency filtering with very low back pressure for high performance. Furthermore, by stacking progressively smaller filters in series, various sizes of beads can be isolated for highly multiplexed assays.

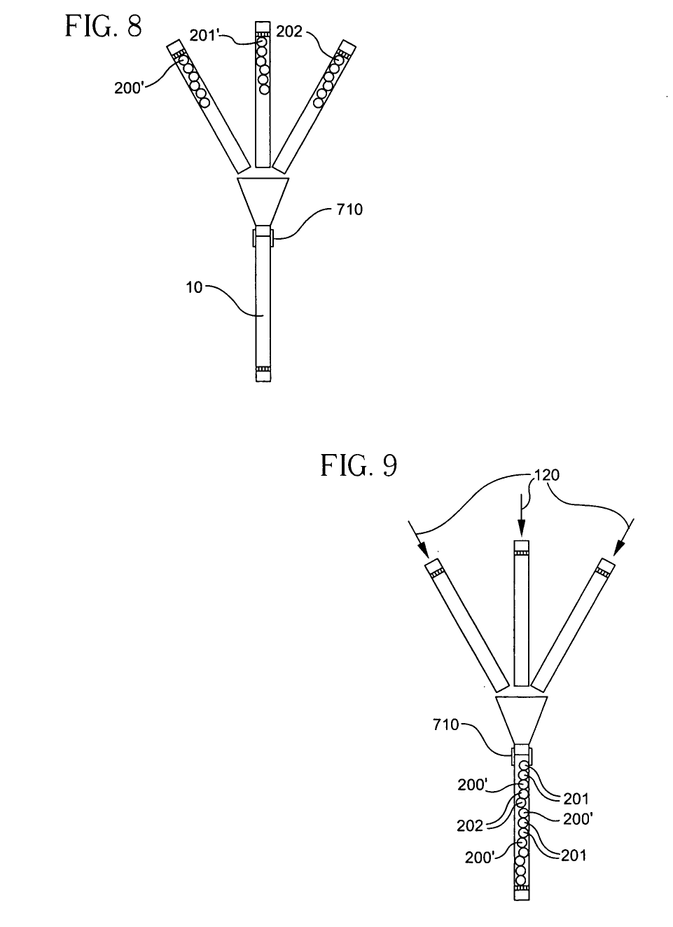

Referring to FIGS. 7-9, different chemistries and different probe immobilizations can be performed separately first with different flavors of particles or differently fluorescing rare-earth beads 200 when performing a multiplex assay with the filtered capillary 10. After finishing the immobilization step of FIG. 7, the particles are recovered and combined into a clean and unused filtered capillary 10 of FIG. 8 by injecting a wash solution on the opposite end of each filtered capillary 10. Next, a solution 120 with different targets can be injected into the filtered capillary 10 containing the combined particles 201, 200′, and 202 in FIG. 9. At the end of the assay, the particles can be washed and imaged, one particle at a time or the whole sequence at one time.

Referring to FIG. 4, a simplified representation of stacking or otherwise bundling three individual filtered capillary tubes, each serving as a separate microfluidic reactor 10, acting in a multiplexed fashion is shown. More tubes can be bundled together but for drawing simplicity, only three are shown. Instead of using the same microfluidic reactor 10 for bio-assaying all the different microbeads or particles 200, if the optical detection system does not allowing detection of such an arrangement of multiple beads, then the beads 200 can be separated into individual microfluidic reactor 10 in a bundled arrangement as shown in FIG. 4.

Using more than one integrated capillary filtered tube can provide a high throughput particle-based assay device, according to the teachings of the present invention. Preferably, the inner height 40 of the square or rectangular capillary filter tubes should be less than twice the height of the particles such that only a monolayer of particles can be formed inside the capillary tubes when the tubes are bundled together. In this case, an inlet interface shaped as a funnel can be used to inject the particles into the tube, as seen in FIGS. 7-9. The filter inside each of the capillary tube has the capability of isolating particles without any additional fluid handling or transferring inside each of the capillary tubes but also can be adapted to a high throughput format. By thus eliminating fluid handling and transferring, the teachings of the present invention solves the problems associated with particle loss and stacking, and hence reduces the number of particles required and increases the quality of the imaged results. Also, the amount of sample or assay mixture required can be significantly reduced which can potentially provide a huge cost savings with reduction in sample and particles consumption. With the capability of adapting to a high throughput format, the present invention has overcome the low throughput barrier.

Referring to FIGS. 7-9, an injection interface 700 can optionally be used with the filtered tube 10, in accordance with the teachings of the present invention. The injection interface 700 can be a separate part or an integral part of the filtered tube 10 to help provide the flow inlet. The injection interface 700 is preferably shaped as a funnel and can be used to inject the particles 200 of FIG. 1 into each of separately formulated filtered tube 10 to form the particles 200′, 201, and 202 having different chemistries and immobilizations.

As in FIG. 1, the filtered capillary 10 has an inner height less than twice the height of the particles 200 and preferably is less than 60 μm when the particle is about 30 μm, for example.

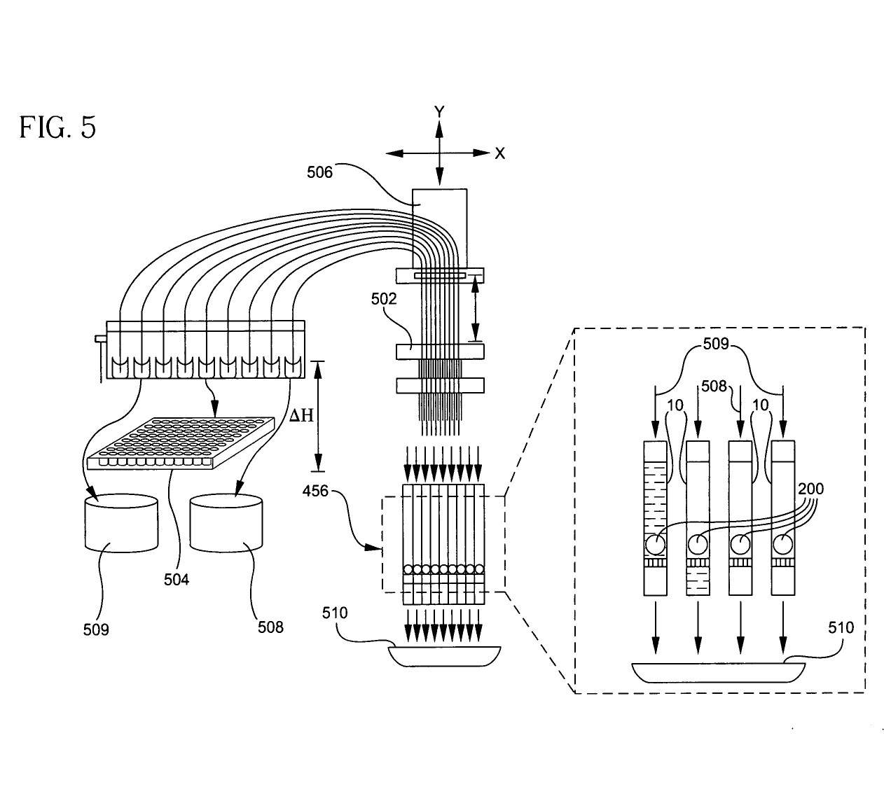

The interface 700 can be made of a glass, polymer or metal. Preferably, the wider mouth of the funnel-shaped interface 700 widens from, for example, about 0.05 mm to about 5 mm to form a funnel shaped inlet. The interface 700 also includes a sleeve 710 made of polymer, metal, or any other suitable support. The sleeve 710 has a polymer inside a coating or other similar material such that a water tight seal is formed between the interface 700 and the filtered capillary 10 when the filtered capillary 10 is plugged into the interface 700. Automated fluid dispensing system, such as the system seen in FIG. 5, can then be used to inject the particle solution 20 into the filtered capillary 10. If particles 200 are stuck at the inlet of the interface 700, the particle solution 120 can be pumped up and down to loosen the particles 200 until the particles 200 are forced into the filtered capillary 10 to form a monolayer.

Referring to FIG. 5, a high throughput system is shown using the bundled filtered capillary tubes of FIG. 4. A high throughput system can result by arranging the square or rectangular capillary filter tubes 10 in parallel and in a monolayer such that the scanning and imaging can be conveniently performed one layer at a time. Preferably, the inner height of the square or rectangular capillary filter tubes should be less than twice the height of the particles such that only a monolayer of particles can be formed inside the capillary tubes. Thus, all the particles of the monolayer can be scanned and imaged simultaneously.

Without any complicated fluidic manipulation systems, the present invention can be adapted to the current microplate format. Rectangular capillary filter tubes are held in a reusable holder 502, which has the same footprint as standard microplates 504, for performing the assay. A conventional robotic fluidic handling system 506, having robotic arms for moving in the X, Y and Z directions can be used to carry out the multiple wash cycles 509 and incubations. The reusable holder 502 having the same footprint as standard microplates 504, e.g., 96, 384 and 1536 well plate format, holds the plurality of capillary filter tubes 10 together such that the existing robotic fluidic handling system 506 injects reagent 508 and sample from the well 504 into the flow inlet of individual capillary filter tubes 10. The holder 502 will allow an easy and convenient exchange or replacement of individual capillary filter tubes, if one is broken or needs to be modified for another surface chemistry.

Each of the filtered capillary tubes 10 is filled with one carrier support bead 200 as the particle that preferably is encoded with at least one rare-earth florescent dopant in a glass host. Numerous wash cycles 509 and incubations with minimal loss of the glass beads can next be performed. A sample is formed by disposing different types of probes contained in the well with a reagent 508. The sample containing the different types of probes for attaching to the bead are poured in each tube to form a particle mixture. Any unattached probes in a solution in each tube are filtered-out into a waste reservoir 5 10. Different labeled targets for binding with the attached probe in each tube are poured into each tube. All unbinded labeled targets in each tube are filtered-out. As seen in FIG. 4, the unbinded or otherwise free targets 300 associated with their labels 100 marked 3, 4, and 8 are suspended in the solution that flows out of the porous filter. In between each of the steps, wash 509, dry, and incubation can optionally be added to purify the sample to be assayed. At the completion of the assay, the particle mixture can be directly injected into a cytometer for performing data analysis. Alternatively, light shining in the direction into the paper (Z direction) can image or otherwise detect the beads with individually attached probes binded with labeled targets. The tubes are left aligned parallel, next to each other, in a planar array to fit the image optical head of the detector in the direction into the paper (Z direction).

Instead of arraying the filtered capillary tubes in a vertical (Y) position for detection, a monolayer of the filtered capillary tubes can be laid horizontally to also fit the image optical head of the detector pointing either on the top or bottom of the horizontal monolayer of filtered capillary tubes.

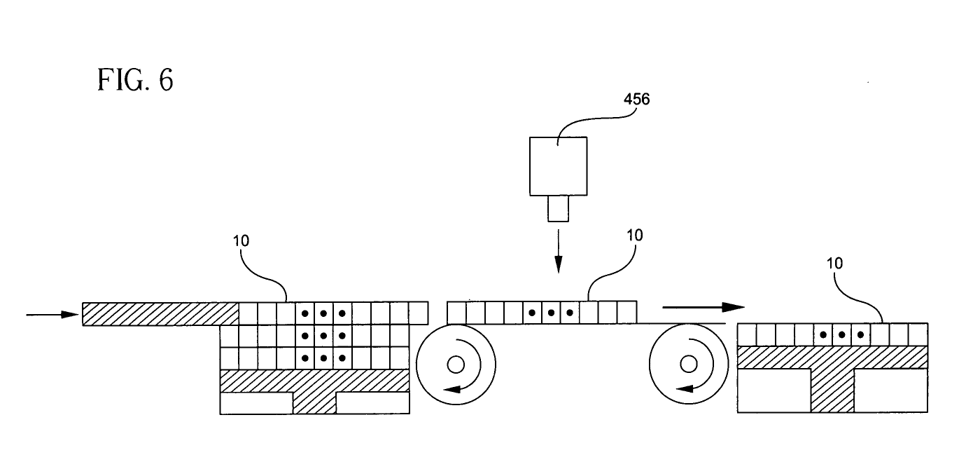

Referring to FIG. 6, a side-view of an automated high throughput particle-based assay system for a horizontal placement during data scanning and imaging is represented. At the completion of the assay of FIG. 5, the holder which carries the rectangular capillary filter tubes can be turned side ways and the tubes 10 are loaded into a loading container of the automated system of FIG. 6. The first layer of the rectangular capillary filter tubes is pushed up by a first piston (Step 1) and a second piston pushes the tubes forwards (Step 2) to a conveyor belt which carries the tube (Step 3) to a data analysis system (Step 4). Finally, the analyzed capillary filter tubes are dumped into an unloading container for storage (Step 5).

Optionally, reagents can be injected into the capillary filter tubes from a horizontal position while the capillary filters are on the conveyor belt, if the reagents would not flow backwards. Otherwise, back pressure was applied, or the capillary filtered tubes were slanted slightly downward. Hence, an interface for each capillary filter tube can be adapted for easy and convenient loading or unloading of reagents and samples.

In an alternate configuration of the automated high throughput particle-based assay system, the whole system would be rotated 900 (i.e., FIG. 6 would be the top view of the system, representing FIG. 5 and the imaging plane 456 of FIG. 4, instead of the side view, at either opposed sides, and imaging plane 456′ which can be for the top or the bottom of the filtered capillary tube in FIG. 4). In other words, the capillary filter tubes will be lining up and standing in a vertical position and the conveyor belt will move the capillary filter tubes to the data analysis system which is in a horizontal position perpendicular to the capillary filter tubes. Then, if one desires, reagents can be easily injected into the capillary filter tubes from the top or bottom.

In summary, the present invention is a high throughput biological particle-based assay device for allowing particle-based assays to be performed with minimal sample, minimal particle loss or human handling and enables the particles to be imaged or scanned at the completion of the assay without any additional fluid handling or transferring. Also, data analysis can be performed within the device without additional fluid handling and transferring. Unlike current technologies that require complicated fluidic handling system to manipulate the particles, the present invention isolates the particles within the same device to form a monolayer for easy scanning and/or imaging, and can be adapted to a high throughput format.

It will be apparent to those skilled in the art that various modifications and variations can be made to the present invention without departing from the spirit and scope of the invention. Thus it is intended that the present invention cover the modifications and variations of this invention provided they come within the scope of the appended claims and their equivalents.

1. A microfluidic reactor for trapping one or more particles of predetermined nominal size or range of sizes, comprising:
a flow inlet;
a transparent capillary for providing an in-situ zone for analysis; and
a porous filter integrated with the transparent capillary, the filter having a plurality of holes defined therein, the holes being smaller than the nominal size or range of sizes and arranged so as to trap the particles in the analysis zone while a fluid flows from the flow inlet through the analysis zone and the filter.

2. An apparatus as claimed in claim 1 wherein the filter extends laterally across the analysis zone.

3. An apparatus as claimed in claim 1 wherein the flow inlet defines a flow axis and the filter intersects the flow axis so as to form a porous reaction chamber.

4. An apparatus as claimed in claim 3, wherein the holes of the porous reaction chamber is substantially hexagonal.

5. An apparatus as claimed in claim 1 wherein the holes are defined between walls of a plurality of small capillaries smaller than the transparent capillary.

6. An apparatus as claimed in claim 5 wherein the plurality of small capillaries are substantially parallel.

7. An apparatus as claimed in claim 6 wherein the transparent capillary comprises at least one rectangular tube to form a planar surface.

8. An apparatus as claimed in claim 1, wherein the transparent capillary is made from glass.

9. An apparatus as claimed in claim 1, wherein the transparent capillary is made from a polymer.

10. An apparatus as claimed in claim 1, wherein the transparent capillary is coated with a solvent resistance.

11. An apparatus as claimed in claim 5, wherein the transparent capillary is heated with the plurality of small capillaries in a collapsed region.

12. An apparatus as claimed in claim 1, wherein the smallest dimension of the transparent capillary is smaller than the size of two particles.

13. An apparatus as claimed in claim 1, further comprising a manipulation system for moving more than one microfluidic reactor in a high throughput bio-assay operation.

14. A microfluidic reactor for trapping one or more particles of predetermined nominal size or range of sizes, comprising:
a flow inlet;
a transparent capillary for providing an in-situ detection zone wherein the detection zone is arranged so as substantially to correspond in shape to an optical detector; and
a porous filter integrated with the transparent capillary, the filter having a plurality of holes defined therein, the holes being smaller than the nominal size or range of sizes and arranged so as to trap the particles in the detection zone while a fluid flows from the flow inlet through the detection zone and the filter.

15. The reactor of claim 14, wherein the particles comprise microbeads.

16. The reactor of claim 14, wherein the optical detector comprises a charge-coupled device for detecting light coming from the reaction in the detection zone.

17. A method for trapping one or more particles of predetermined nominal size or range of sizes, comprising the steps of:
providing a flow inlet;
providing an in-situ transparent analysis zone;
integrating a porous filter with the in-situ transparent analysis zone, the filter having a plurality of holes defined therein, the holes being smaller than the nominal size or range of sizes;
flowing a fluid from the flow inlet through the analysis zone; and
trapping the particles in the analysis zone while the fluid flows through filter

18. The method of claim 17, wherein the flowing step comprises reacting the fluid having an analyte with a probe immobilized on a plurality of particles.

19. The method of claim 17, wherein the flowing step comprises flowing a fluid of whole blood cells.

20. The method of claim 18 further comprising scanning the trapped particles for a visible result of the reaction in the detection zone.
